(19) United States
(12) Patent Application Publication
(10) Pub. No.: US 2026/0286281 A1
(43) Pub. Date: Sep. 24, 2026

(54) METHOD FOR DEGRADING GLYCOGEN PRESENT IN A BIOMASS OF GALDIERIA

(71) Applicant: FERMENTALG, Libourne (FR)

(72) Inventors: Olivier CAGNAC, St Martin du Bois (FR); Axel ATHANE, Carbon Blanc (FR); Matthieu COURBALAY, ALLONNES (FR)

(21) Appl. No.: 19/475,490

(22) PCT Filed: Apr. 19, 2024
(86) PCT No.: PCT/EP2024/060721

§ 371 Date: Oct. 15, 2025

(30) Foreign Application Priority Data
Apr. 19, 2023 (FR) FR2303894

(51) Int. Cl. C12N 1/06; A61K 8/9717; A61K 36/04; A61Q 19/00

(52) U.S. Cl. CPC C12N 1/06; A61K 8/9717; A61K 36/04; A61Q 19/00

(57) ABSTRACT

The present invention relates to a treatment method for the degradation of the glycogen present in a biomass of unicellular red algae (URA), particularly of the genus Galdieria, and to the product thus obtained.

### FIELD OF THE INVENTION

The present invention relates to a treatment method for the degradation of the glycogen present in a biomass of unicellular red algae (URA), particularly of the genus Galdieria, and to the aqueous product thus obtained.

### PRIOR ART

Unicellular red algae (URA), or Rhodophytes, are characterized by the presence of pigments within their cells. In addition to chlorophyll and carotenoids, unicellular red algae produce phycobiliproteins. These natural pigments derived from photosynthesis are divided into four types: allophycocyanins, C-phycocyanins, phycoerythrins and phycoerythrocyanins.

Phycocyanins have beneficial properties for human and animal health, and are now used in a wide range of fields, including pharmaceuticals, cosmetics and the food industry.

Microalgae belonging to the Cyanidiophyceae class, and more specifically to the genera Cyanidioschyzon, Cyanidium and Galdieria, are particularly interesting for phycocyanin production. The production of biomasses from unicellular red algae is well known to those skilled in the art, particularly for the production of molecules of interest, notably proteins such as phycocyanins. Methods for producing and extracting said phycocyanins are described in the literature (WO2017/093345, WO2019/228947, WO2018/178334, WO2020/161280).

However, microalgae of this class, notably the genus Galdieria, are also known to have glycogen as a major reserve sugar. This glycogen is a polymer of α(1→4) glucoses branched by α-(1→6) linkages. Glycogen from microalgae of the genus Galdieria has the particular feature of possessing a high proportion of these branches, that is around 7 to 18% of branched glucoses randomly distributed along the molecule. This molecular structure gives glycogen a globular form, making it soluble in water (Martinez-Garcia et al., Int J Biol Macromol. (2016) 89:12-8). Moreover, if microalgae of the genus Galdieria possess the enzymes for the synthesis of highly branched glycogen, they also possess the enzymes to degrade it (Martinez-Garcia et al., Int J Biol Macromol. (2016) 89:12-18). These enzymes are intracellular enzymes. The intracellular medium of microalgae in this class has a pH of between 6.3 and 7.1 (Miyagishima et al., Plant Cell Physiol. 62(6): 926-941 (2021)). These enzymes are therefore known to be active in this precise pH range.

For the preparation of aqueous extracts from microalgae, such as phycocyanin extracts from Galdieria sulphuraria, filtration steps may be necessary. As glycogen is easily soluble in cold water, it is found in the aqueous fraction along with hydrophilic compounds of interest, such as phycocyanins. The filters used to purify phycocyanins retain all or part of the glycogen, thus increasing the viscosity of the retentate. This creates technical constraints on the method, such as problems with pressure build-up, flow reduction and clogging, particularly with tangential filtration membranes. On the other hand, if the glycogen is not eliminated, the prepared extract will be high in glycogen, viscous and low in phycocyanins.

Glycogen is known to be resistant to certain enzymes. However, a method involving the addition of suitable exogenous enzymes for its degradation has already been developed (WO2020/144330).

If it cannot be completely eliminated and/or inactivated, an enzyme added for the preparation of a dietary product must be listed in its composition. Such a non-eliminable substance corresponds to an additive or processing aid which may pose marketing and/or formulation difficulties.

Other methods for preparing aqueous extracts from biomasses of unicellular red algae, particularly of the genus Galdieria, have been described in the literature (Moon et al., Korean J. Chem. Engl. 2016, 31, 3, pages 490-495). However, these methods are difficult to apply on an industrial scale, since they require either the addition of large quantities of ammonium sulfate, resulting in effluents rich in pollutants that are undesirable for ecological and economic reasons, or the use of very expensive instruments such as chromatographs, or a sequence of steps that are difficult to implement on a larger scale than the laboratory. Furthermore, none of these methods can selectively remove glycogen from biomass or aqueous microalgae extracts.

There is therefore a need to provide a method for degrading glycogen present in a biomass of unicellular red algae whose main reserve sugar is glycogen, in particular in a biomass of microalgae of the genus Galdieria, while overcoming the problems of the prior art, and in particular those set out above.

### DISCLOSURE OF THE INVENTION

According to a first aspect of the invention, the inventors have developed a method for treating a biomass of unicellular red algae (URA) of the genus Galdieria, said URA biomass treatment method comprising the steps of:
a) harvesting of biomass by separating the culture medium to obtain a raw biomass of URA,
b) cell lysis of the crude biomass from step (a) to obtain a lysate,
c) optional dilution of the lysate from step (b) to obtain a solubilizate, and
d) separation of insolubles suspended in the lysate from step (b) or the solubilizate from step (c) to obtain a clarified fraction, the method comprising a step of glycogen degradation in which the raw biomass and/or lysate and/or solubilizate is kept at rest for at least 3 hours, during which the liquid medium comprising glycogen is at acidic pH.

Surprisingly, glycogen degradation by the microorganism's endogenous enzymes is effective over a wide pH range not limited to the intracellular pH of said microorganism, with an optimum for more acidic pHs, notably between 2 and 5. Still surprisingly, enzymatic degradation works inside the cell in processed biomass such as raw biomass, thawed raw biomass or dried raw biomass, but also in lysate and solubilizate. The present invention further relates to a product obtainable by a method according to the invention.

Advantageously, the method according to the present invention makes it possible to reduce or even eliminate the addition of exogenous enzymes adapted for the degradation of ARU glycogen, thus reducing the addition of substances that must be qualified as additives and/or processing aids.

The method according to the invention can also be applied under conditions of time, temperature and pH ranging from the mildest to the most stringent, and can therefore be adapted to the stability of the molecules of interest to be extracted.

### DESCRIPTION OF THE FIGURES

FIG. 1 shows free glucose concentrations in Galdieria sulphuraria biomass lysates over time at pH 3.75 and pH 6, at 37° C. without the addition of exogenous enzymes (SE: Without Enzyme) and with 1% exogenous enzymes in the lysate (E: Enzyme).

FIG. 2 shows free glucose concentrations in Galdieria sulphuraria biomass lysates at room temperature (“amb”, that is 20° C.) in the absence of exogenous enzymes and at different pH levels as a function of time.

FIG. 3 shows free glucose concentrations in Galdieria sulphuraria biomass lysates at pH 3.75 at different temperatures, in the presence or absence of exogenous enzymes, as a function of time (WE: Without Enzyme; E: Enzyme).

FIG. 4 shows free glucose concentrations in Galdieria sulphuraria biomass lysates at pH 3.75 at different temperatures, in the absence of exogenous enzymes, as a function of time.

FIG. 5 shows free glucose concentrations as a function of time in Galdieria sulphuraria biomass lysate and solubilizate at pH 3.75 and 20° C. in the absence of exogenous enzymes, and in Galdieria sulphuraria biomass lysate at pH 3.75 in the presence of exogenous enzymes (WE: Without Enzyme; E: Enzyme).

FIG. 6 shows the percentage of glycogen digestion as a function of time in Galdieria sulphuraria biomass clarified at pH 4 and 20° C., in the absence or presence of exogenous enzymes.

FIG. 7 shows the percentage of glycogen digestion as a function of time in Galdieria sulphuraria biomass lysate, solubilizate and clarified fraction at pH 4 and 20° C. in the absence of exogenous enzymes (WE: Without Enzyme).

### DETAILED DESCRIPTION OF THE INVENTION

### Definitions

In the context of the present invention, the term “biomass” refers to a collection of microalgae cells, preferentially produced by fermentation in a biological reactor. Said biomass can be seen as a mass of single-celled organisms.

The biomass can undergo various treatments and be raw biomass, lysed biomass, thawed raw biomass and/or dried raw biomass.

It is understood in the context of the present application that the properties of the biomass correspond to the average of the properties of all the cells making up said biomass, in other words, a lysed biomass is a biomass comprising at least 50% of lysed cells in relation to the total number of cells, and a raw biomass may comprise lysed cells due to the harvesting step without being considered a lysed biomass as long as the number of lysed cells over the number of non-lysed cells remains a minority, that is less than 50%.

The term “raw biomass” refers to biomass obtained after harvesting, that is after recovery of the fermentation broth and then separation of the cells from at least part of the culture medium, possibly thawed and/or dried.

The expression “lysed biomass” or “lysate” refers to a microalgae biomass in which at least 50% of the cells are lysed, preferentially at least 70%, more preferentially in which at least 80%, 85%, 90%, 95%, up to 100% of the cells are lysed.

The expression “thawed raw biomass” refers to raw biomass that has been frozen, possibly for storage and/or transport reasons, and then thawed to reach a temperature suitable for processing according to the invention, in particular a temperature suitable for the step of glycogen degradation of the biomass according to the invention.

According to the invention, the expression “dried raw biomass” refers to raw microalgae biomass that has been dried using methods known to the skilled person and whose water content relative to the total weight of the biomass is less than 10%, preferentially less than 7%, more preferentially between 5% and 1% water. Known drying methods include natural air drying, spray drying, fluidized air bed drying, roller dryer drying and freeze drying.

The term “solubilizate” refers to a lysed biomass or lysate that has undergone a step of dilution with an aqueous solution of neutral, acidic or basic pH.

The term “clarified fraction” refers to an aqueous extract obtained after separation of insolubles suspended in a lysate or solubilizate.

The term “rest” refers to a stage during which the chemical properties of the preferentially dried or frozen raw biomass, lysate and/or solubilizate is not modified by adding or extracting one or more component(s). Rest does not preclude the raw biomass, lysate, solubilizate and/or clarified fraction from being mixed, that is stirred, under non-destructive conditions that do not affect the chemical properties of the raw biomass, lysate and/or solubilizate.

The term “liquid medium comprising glycogen” describes the intracellular medium of the cells making up the raw biomass and/or the liquid phase of the lysate, solubilizate and/or clarified fraction, where applicable.

The term “biomass treatment” refers to any method applied to a biomass, in particular to modify its physical/chemical properties, extract molecules of interest and/or purify them.

Note that the numerical intervals given are intended to include all intermediate numbers (for example, an interval from 1 to 5 includes 1, 1.5, 2, 2.75, 3, 3.80, 4, 4.32 and 5).

Please note that all numerical values given refer to the actual value given, as well as to approximations of this value estimated on the basis of the general convention that the last digit indicated corresponds to the precision of the measurement. In the absence of precise error limits, the maximum error for the last digit specified should be estimated using the rounding convention.

### Treatment Method According to the Invention

The method according to the invention is a method for treating a biomass of unicellular red algae (URA) of the genus Galdieria, said URA biomass treatment method comprising the steps of:
a) harvesting of biomass by separating the culture medium to obtain a raw biomass of URA,
b) cell lysis of the crude biomass from step (a) to obtain a lysate,
c) optional dilution of the lysate from step (b) to obtain a solubilizate, and
d) separation of insolubles suspended in the lysate from step (b) or the solubilizate from step (c) to obtain a clarified fraction,
the method comprising a step of glycogen degradation in which the raw biomass and/or lysate and/or solubilizate is kept at rest for at least 3 hours, during which the liquid medium comprising glycogen is at acidic pH.

Ideally, throughout the method, temperature, time and pH conditions are particularly adapted so as not to degrade the compounds of interest in the aqueous extract, e.g. phycocyanins, while promoting glycogen degradation during the resting stage.

In one embodiment, the step of glycogen degradation is performed on the raw biomass. This embodiment then comprises, in order, the steps of:
a) harvesting of biomass by separating the culture medium to obtain a raw biomass of URA,
the raw biomass rests for at least 3 hours, during which the liquid medium comprising glycogen is at acidic pH,
b) cell lysis of the crude biomass from step (a) to obtain a lysate,
c) optional dilution of the lysate from step (b) to obtain a solubilizate,
d) separation of insolubles suspended in the lysate from step (b) or the solubilizate from step (c) to obtain a clarified fraction, and optionally, a step of adjusting the pH of the raw biomass.

In this embodiment, the glycogen is in the intracellular medium of the cells constituting the biomass, which liquid medium is at an acidic pH, and the raw biomass is preferentially a dried raw biomass or thawed raw biomass. In the next two embodiments, after cell lysis, the glycogen is found in the liquid phase of the lysate and, where applicable, the solubilizate.

In one embodiment, the step of glycogen degradation is performed on the lysate. This embodiment then comprises, in order, the steps of:
a) harvesting of biomass by separating the culture medium to obtain a raw biomass of URA,
b) cell lysis of the crude biomass from step (a) to obtain a lysate, resting the lysate for at least 3 hours in a liquid medium containing glycogen at acidic pH,
c) optional dilution of the lysate from step (b) to obtain a solubilizate,
d) separation of insolubles suspended in the lysate from step (b) or the solubilizate from step (c) to obtain a clarified fraction, and optionally, a step of adjusting the pH of the raw biomass or of the lysate.

It may be necessary to adjust the pH of the liquid medium of the raw biomass and/or lysate to obtain the acidic pH required for the step of lysate rest.

In another embodiment, the step of glycogen degradation is performed on the solubilizate. This embodiment then comprises, in order, the steps of:
a) harvesting of biomass by separating the culture medium to obtain a raw biomass of URA,
b) cell lysis of the crude biomass from step (a) to obtain a lysate, c) dilution of the lysate from step (b) to obtain a solubilizate, the solubilizate rests for at least 3 hours, during which the liquid medium comprising glycogen is at acidic pH,
d) separation of insolubles suspended in the solubilizate from step (c) to obtain a clarified fraction, and
optionally, a step of adjusting the pH of the raw biomass, of the lysate, or of the solubilizate.

It may be necessary to adjust the pH of the liquid medium of the raw biomass, of the lysate, and/or of the solubilizate to obtain the acidic pH required for the step of solubilizate rest.

### a) Harvesting the Biomass

The biomass according to the invention is a biomass of unicellular red algae (URA), more particularly a biomass of phycocyanin-producing microalgae with a high glycogen content.

These microalgae belong to the Cyanidiophyceae class, which includes the genera Galdieria, Cyanidium and Cyanidioschyzon. Preferentially, microalgae are of the genus Galdieria.

Microalgae of the Galdieria genus include Galdieria daedala, Galdieria maxima, Galdieria partita, Galdieria sulphuraria, Galdieria phlegrea, Galdieria javensis, Galdieria yellowstonensis, Galdieria sp Preferentially, the biomass according to the invention is a biomass of Galdieria sulphuraria.

Methods for producing biomass of unicellular red algae (URAs), and in particular from the genus Galdieria, are well known to the skilled person. In the context of the present invention, microalgae can be cultivated using any known cultivation technique, in containers suitable for growing microorganisms, also known as biological reactors, bioreactors or fermenters.

According to the invention, biomass of unicellular red algae is obtained from microalgae cultivated industrially in a high-capacity reactor, preferably to obtain fermentation musts comprising high densities of microorganisms. For the purposes of this application, “high densities” means a quantity corresponding to more than 50 g of dry matter per liter of fermentation broth, preferentially more than 100 g per liter. Examples of single-cell red algae cultures are described in patent applications (WO2017/050917, WO2017/050918, WO2017/093345 and WO2019/228947). It is understood that the skilled person will be able to determine the optimal parameters and conditions for cultivating microorganisms, such as temperature, lighting, cultivation time or the nature and quantity of nutrients to be supplied. The method according to the invention includes a step a) of harvesting biomass.

Once the microorganisms have been cultivated and the biomass obtained, it is harvested to produce raw biomass. Unicellular red algae can be harvested using any technique known to the skilled worker, including filtration, possibly gravimetric or under reduced pressure, decantation, precipitation followed by gravimetric filtration, or centrifugation.

The method according to the invention comprises a step a) of harvesting biomass corresponding to the recovery of the fermentation broth, followed by the biomass cells separating from at least part of the culture medium.

This step produces a raw biomass. The raw biomass harvested in this way can also be washed, preferably with water, to remove certain soluble impurities.

The raw biomass obtained after harvesting and optionally after one or more washing(s) comprises at least 70% water, and up to 90% water, preferentially it comprises from 75 to 88% water.

Preferentially, the raw biomass according to the invention has a dry matter content of 5 to 30% by weight relative to the total weight of the raw biomass, generally still preferentially 10 to 25% by weight, more preferably 10 to 20% by weight.

Also preferentially, the crude biomass according to the invention has a C-phycocyanin concentration of between 3 and 12%.

### b) Cell Lysis

The method for treating unicellular red algae biomass according to the invention comprises a step b) of cell lysis of the raw biomass from step a) to obtain a lysate.

Preferentially, step b) of cell lysis is carried out on a raw biomass with a dry matter content of 5 to 30% by weight relative to the total weight of the raw biomass, preferentially 10 to 25% by weight, more preferentially 10 to 20% by weight.

Also preferentially, step b) of cell lysis is carried out on a crude biomass according to the invention with a C-phycocyanin concentration of between 3 and 12%.

Cell lysis can be carried out by any method of lysis known to the skilled worker, in particular by enzymatic, mechanical and/or chemical means.

In the context of the invention, prior to this step b) of cell lysis, the raw biomass may have undergone a step of washing, freezing, thawing, drying and/or rehydration. In other words, in the context of the present invention, raw biomass can be thawed and/or dried biomass.

In the preferred case of mechanical lysis, mechanical means that can be used according to the invention include ball mills, high-shear mixers, high-pressure homogenizers, pin mills, impact mills, ultrasonics, or pulsed electric fields. As devices for implementing these methods, we refer to the ball mill: Discus-100 from Netzsch or ECM-AP60 from WAB, the high-pressure homogenizer: Ariete from GEA, the high-shear mixer: 700-X from Silverson, the pin mill: Contraplex from Hosokawa and the impact mill: Condux from Netzsch.

Preferentially, cell lysis is carried out by mechanical lysis, preferentially by grinding and even more preferentially with a ball mill.

Preferentially, the lysate obtained has a dry matter content of 5 to 30% by weight relative to the total weight of the lysate, preferentially 10 to 25% by weight, more preferentially 10 to 20% by weight.

Also preferentially, the lysate obtained has a C-phycocyanin concentration of 3% to 12% relative to the total weight of dry matter.

This step b) of lysis can be carried out before or after the step of glycogen degradation, that is before or after the step of rest, preferentially before.

### c) Optional Lysate Dilution

According to the invention, the lysed biomass or lysate can optionally undergo a step of dilution.

In the context of the present invention, the step of dilution refers to the addition of a solution to the lysed biomass or lysate to reduce its dry matter concentration.

The diluted lysed biomass is then defined as a “solubilizate”.

Advantageously, the step of dilution is performed by adding an aqueous solution.

In one embodiment, the aqueous solution is water.

Preferentially, the step of dilution c) is carried out on lysate with a dry matter content of 5 to 30% by weight relative to the total weight of the lysate, preferentially 12 to 25% by weight.

Also preferentially, the step of dilution c) is carried out on lysate with a C-phycocyanin concentration of 3% to 12% relative to the total weight of dry matter.

The aqueous solution may additionally comprise one or more pH-adjusting compounds. The term “pH-adjusting compounds” refers to any organic or inorganic compound used to modify pH (acidity correctors, acids, bases, neutralizing agents or buffering agents). Examples of such compounds are sulfuric acid, acetic acid, citric acid, phosphoric acid, sodium citrate, potassium lactate, potassium malate, sodium chloride, disodium phosphate and potassium phosphate. The aqueous solution has an acidic or basic pH, depending on the pH-adjusting compound(s) present in the solution.

Preferentially, step c) of diluting the lysed biomass or lysate is carried out with an aqueous solution with a pH less than or equal to 8, in particular between 0 and 6, preferentially between 1 and 6, more preferentially between 2 and 5. The pH of the aqueous solution to be added to the lysed biomass or lysate can be around 2, around 3, around 4 or around 5. Examples of acid solutions that can be added to lysed biomass are solutions comprising acids such as those described in the previous paragraph.

Depending on the case, the solubilizate has a pH close to neutrality, with a pH between 6 and 8, a pH between 1 and 6 or a pH between 8 and 14.

Preferentially, the solubilizate has a pH below 7, in particular between 1 and 6, more preferentially between 2 and 5, even more preferentially between 3 and 4.

Preferentially, the solubilizate obtained has a dry matter content of 1 to 15% by weight relative to the total weight of the solubilizate, preferentially 3 to 12% by weight, more preferentially 4 to 8% by weight.

Also preferentially, the solubilizate obtained according to the invention has a C-phycocyanin concentration of 0.1 to 12%, preferentially 0.5 to 8%, more preferentially 1 to 7%.

This step c) of dilution can be carried out before or after the step of glycogen degradation, that is before or after the step of rest.

Preferentially, the step c) of dilution is carried out after the step of rest.

The method according to the invention thus comprises, according to this preferred embodiment, in order, the successive steps of:
a) harvesting of biomass by separating the culture medium to obtain a raw biomass of URA,
b) cell lysis of the crude biomass from step (a) to obtain a lysate, resting the lysate at acidic pH for at least 3 hours,
c) diluting the lysate to obtain a solubilizate, and
d) separation of insolubles suspended in the solubilizate from step (c) to obtain a clarified fraction.

### d) Separating the Insolubles

As part of the method according to the invention, the lysate or solubilizate undergoes a step d) to separate the suspended insolubles to obtain a clarified fraction.

This step (d) of separating the insolubles is carried out after the step of glycogen degradation, that is after the step of rest.

The step of separating the insolubles d) from the lysate or solubilizate can be carried out by any method known to the skilled person. These include frontal filtration and centrifugation.

According to the invention, step d) for separating insolubles is carried out on a lysate with a dry matter content of 5 to 30% by weight relative to the total weight of the lysate, preferentially 10 to 25% by weight, more preferentially 15 to 20% by weight; or more preferentially on a solubilizate with a dry matter content of 1 to 15% by weight relative to the total weight of the solubilizate, preferentially 3 to 12% by weight, more preferentially 4 to 8% by weight.

Preferentially, according to the invention, step d) for separating insolubles is carried out on a lysate or solubilizate with a pH of less than 7, preferentially between 1 and 6, even more preferentially between 2 and 5 and still more preferentially between 3 and 4.

The clarified fraction according to the invention preferentially has a dry matter content of 0.1% to 5% by weight relative to the total weight of the clarified fraction, preferentially 0.5% to 4% by weight, more preferentially 1% to 3% by weight.

The clarified fraction according to the invention preferentially has a C-phycocyanin concentration of 0.1 to 20 g/L. In the particular case where the method according to the invention does not comprise step c), the clarified fraction according to the invention has a C-phycocyanin concentration of 7 to 19 g/L, even more preferentially 12 to 18 g/L. In the opposite case, where the method according to the invention comprises a step c), the clarified fraction according to the invention has a C-phycocyanin concentration of 0.1 to 12 g/L, preferentially 0.5 to 8 g/L, more preferentially 1 to 7 g/L.

### Glycogen Degradation

The method according to the invention is characterized by a step of glycogen degradation using the endogenous enzymes of the microorganism, by keeping the raw biomass and/or lysate and/or solubilizate at rest (step of rest) for at least 3 hours, during which the liquid medium comprising glycogen is at acidic pH.

In order for glycogen degradation by the microorganism's endogenous enzymes to be effective during the step of rest, the endogenous enzymes must be active and therefore must not have been inactivated, for example by a step of heating step prior to said step of rest.

### Moment of the Step of Rest

Resting for at least 3 hours is carried out at acidic pH, on raw biomass, lysate and solubilizate. In particular, according to the invention, resting can be carried out before or after cell lysis step b), that is on raw biomass, lysate and/or solubilizate.

According to the invention, the step of rest can advantageously be carried out under agitation of the raw biomass and/or the lysate and/or the solubilizate.

Preferentially, the step of rest is carried out only on raw biomass which is preferentially dried or thawed, lysate, or solubilizate. In the case of lysate and/or solubilizate, the step of rest is carried out if necessary after adjusting the pH to obtain the acidic pH required for the step of rest.

Preferably, according to the invention, resting is carried out after cell lysis step b), in other words, preferably on the lysate and/or solubilizate and even more preferably on the lysate.

Preferably, the step of rest is carried out on a medium comprising lysed cells, that is on the lysate from step b) and/or, where applicable, on the solubilizate from step c).

### Conditions of the Step of Rest

According to the invention, the step of rest can be carried out on raw biomass, lysate and/or solubilizate immersed in the dark or exposed to natural or artificial light. Preferably, the step of rest is carried out on raw biomass, lysate or solubilizate immersed in the dark.

According to the invention, the step of rest is performed on raw biomass, lysate or solubilizate, with or without aeration.

The “acidic pH” of the glycogen-containing medium during the resting stage is defined as a pH below 7, in particular between 1 and 6, preferably between 2 and 5, even more preferably between 3 and 4.

For raw biomass, the glycogen is in the intracellular medium of the cells making up the biomass, which is a liquid medium at an acidic pH.

Optionally, the method according to the invention further comprises a step of adjusting the pH of the raw biomass, lysate and/or solubilizate to the acidic pH of the step of rest.

In this case, the pH of the raw biomass, lysate and/or solubilizate is adjusted to a pH below 7, in particular between 1 and 6, even more preferably between 2 and 5, and still more preferably between 3 and 4.

Reagents for adjusting pH, that is acidifying or basifying raw biomass, lysate or solubilizate, can be added in solid or solution form. Advantageously, the pH of the raw biomass, lysate or solubilizate is adjusted by adding an acidic or basic solution, preferably in the form of an aqueous solution.

Examples of pH-adjusting compounds are given above in the description of step c): sulfuric acid, hydrochloric acid, nitric acid, phosphoric acid, acetic acid, sodium hydroxide, sodium carbonate or sodium bicarbonate.

It is understood that the skilled person will be able to determine whether an acidic or basic compound or solution needs to be added to the raw biomass, lysate and/or solubilizate in order to adjust the pH to the desired value.

When present, this step of adjusting pH is carried out upstream of the step of rest, on the preferably thawed and/or dried raw biomass, on the lysate or on the solubilizate.

Advantageously, when present, this step of adjusting pH is carried out on the lysate prior to the step of rest and prior to steps c) and d) of dilution and separation.

Alternatively, the step of adjusting pH can be performed concurrently with step of dilution c), if present. In particular, if this step c) is present, the step of adjusting pH can be carried out on the lysate concomitantly with this step of dilution c) prior to the step of rest. In this case, after dilution of the lysate, the solubilizate obtained has a pH below 7, in particular between 1 and 6, preferably between 2 and 5, even more preferably between 3 and 4.

The step of rest can last up to a week or 7 days. Preferably, the step of rest lasts between 3 hours and a week, even more preferably, from 3 hours to 48 hours, even more preferably from 6 to 36 hours and still more preferably from 10 to 24 hours.

According to the embodiment, the rest phase lasts from several hours to several days, in particular around 3 hours, 6 hours, 8 hours, 10 hours, 12 hours, 24 hours, 36 hours, between 48 and 72 hours, between 2 and 7 days, between 3 and 7 days, 4 days, 5 days or 6 days.

According to one embodiment, the temperature of the raw biomass and/or lysate and/or solubilizate during the rest phase is maintained at a temperature of between 15° C. and 70° C., in particular between 15 and 50° C., preferably between 15 and 40° C., even more preferably between 15 and 30° C.

Advantageously, the temperature of the raw biomass and/or lysate and/or solubilizate during this rest phase is maintained at a constant temperature, in particular at a temperature of between 15° C. and 70° C., in particular between 15° C. and 50° C., preferably between 15 and 40° C., even more preferably between 15 and 30° C.

In another embodiment, the temperature of the raw biomass and/or lysate and/or solubilizate during the rest phase is maintained at a temperature below 15° C., preferably between 4° C. and 15° C.

Advantageously, according to this other embodiment, the temperature of the raw biomass and/or lysate and/or solubilizate during this rest phase is kept at a constant temperature, in particular between 4° C. and 15° C. Note that for a step of rest, the closer one approaches the optimal temperature-and-pH combination, the less time is needed for the glycogen to degrade; for instance, for a step of rest at a pH comprised between 3 and 4 and a temperature of between 15 and 30° C., the duration could be between 2 and 10 h, whereas for a step of rest at a pH of around 6 and a temperature of around 10° C., the duration should be between at least 6 and 7 days.

In the context of the present invention, the step of rest is carried out with a liquid medium comprising glycogen free from significant microbiological contamination. Such contamination can be avoided by adding preservatives to the liquid medium comprising glycogen. These preservatives are well known to those skilled in the art and are notably selected from sodium benzoate, potassium benzoate, calcium benzoate, benzoic acid, sodium diacetate, calcium propionate, sodium propionate, sodium nitrate, potassium sorbate, sodium sorbate, methyl gallate, propyl gallate, sodium ethylenediaminetetraacetate, methyl paraben, natamycin, propyl paraben and mixtures thereof.

### Optional Addition of Exogenous Enzymes

The method may further comprise a step of adding exogenous enzymes to complement the step of rest. This addition of exogenous enzymes enables glycogen breakdown to be completed as required, particularly if the desired level of breakdown is not achieved. It is understood that “complement” here means that most, that is at least 50%, of the glycogen degradation is the result of implementing the step of rest, independently of this possible addition.

The term “enzymes” refers to proteins that activate or accelerate chemical or biological reactions. In the context of this application, the term “exogenous enzymes” refers to enzymes that are not naturally produced by the cells or microorganisms of the processed biomass of unicellular red algae (URA). In particular, said exogenous enzymes are selected from enzymes extracted from Aspergillus, Bacillus or Trichoderma.

The exogenous enzymes according to the invention have glycogen degradation activity. These enzymes are well known to the skilled person and are notably selected from enzymes with α1-4 glucuronidase activity, α1-4 glucosidase activity, α1-6 glucosidase activity and amylase activity. Note that it is possible to use any of these enzymes, or a mixture of them. It has been found that these enzymes reduce the size of the glucosidic chains of glycogen present in the medium, which can then be eliminated at a later stage, along with its degradation by-products.

Enzymes with α1-4 glucuronidase and/or α1-4 glucosidase activity include pectinases known to degrade pectin, and in particular pectinases extracted from filamentous fungi such as Aspergillus, and more particularly pectinases extracted from Aspergillus aculeatus, such as the enzymes marketed under the name Pectinex® by Novozymes.

Examples of enzymes with α1-6 glucosidase activity are pullulanases known to hydrolyze pullulan α1-6 glucosidic bonds, which are notably also known to debranch starch. These are generally enzymes extracted from bacteria, notably of the Bacillus genera. Patents U.S. Pat. Nos. 6,074,854 and 5,817,498 and application WO2009/075682 describe such pullulanases extracted from Bacillus deramificans or Bacillus acidopullulyticus. Commercially available pullulanases are also known, notably Promozyme D2 and Novozym 26062 from Novozymes, or Optimax L1000 from DuPont-Genencor.

Among the enzymes with amylase activity known to degrade starch, many are known from the prior art and are described in the literature and in particular patent applications such as WO 2019/036721. Commercially available amylases are known, in particular under the names “Amylase AG XXL” (from Novozymes) or “Panzym® AG XXL” (from Eaton).

The enzymes can be used in their pure or enriched form, and optionally as a mixture with one or more excipients. The enzymes used in the method of the invention are in powder or solution form. In the latter case, the enzymes are preferentially dissolved in water.

Preferred conditions for using exogenous enzymes are a pH below 7 and a reaction temperature below 60° C., preferably below 50° C., and even below 30° C. In particular, the temperature of the solution to which the exogenous enzymes are added and at which the enzymatic reaction takes place is between 4 and 60° C., preferentially between 2° and 42° C., and the pH of the solution is less than or equal to 5, preferably around 4.5. The exogenous enzymes can be added to the medium either in free form or immobilized on a support.

The exogenous enzymes can be added to the lysate, in which case the enzymatic reaction with the exogenous enzymes is carried out on the lysate, while the step of rest is carried out beforehand on the raw biomass and/or beforehand on the lysate and/or concomitantly on the lysate.

In a preferred embodiment, the exogenous enzymes are added after the step of rest. In this case:

When the step of rest is carried out on raw biomass, exogenous enzymes can be added to the lysate and/or solubilizate and/or clarified fraction to complete the glycogen degradation resulting from the resting of the raw biomass.
when the step of rest is carried out on the lysate, exogenous enzymes can be added to the lysate after the step of rest, or to the solubilizer and/or clarified fraction to complete the glycogen degradation resulting from the resting of the lysate;
when the step of rest is carried out on the solubilizate, exogenous enzymes can be added to the solubilizate after the step of rest and/or into the clarified fraction, to complete the glycogen degradation resulting from the resting of the solubilizate;
   According to an even more preferred embodiment, the step of rest is carried out on the lysate and, preferably, any addition of exogenous enzymes is carried out on the clarified fraction or solubilizate.

This combination of resting and adding exogenous enzymes reduces the quantities of exogenous enzymes used compared with prior art methods.

The exogenous enzymes are added in a proportion of less than 0.01% by weight relative to the total weight of the raw biomass, lysate, solubilizate or clarified fraction to be treated, for an enzyme with an enzymatic activity equivalent to that of the Pectinex Ultra SP-L enzyme, with an activity declared by the manufacturer of 3300 PGNU/g.

In one embodiment, the added exogenous enzyme content is less than or equal to 0.005% by weight, preferentially less than or equal to 0.0025%, more preferentially less than or equal to 0.0001% by weight. Note that, if necessary, the concentration of exogenous enzymes is adjusted according to the activity of the exogenous enzyme selected to have an activity in the reaction medium equivalent to that of a concentration according to the invention of Pectinex Ultra SP-L at 3300 PGNU/g.

The skilled person will also know how to adapt the quantities of enzymes to be added during the method in order to increase the degradation of the glycogen present in the lysate, solubilizate and/or clarified fraction.

The step of glycogen degradation, with or without the addition of exogenous enzymes, is considered sufficient, in particular to improve the subsequent filtration step(s), when at least 10%, preferentially at least 50%, of the initial glycogen content in the raw biomass has been degraded, even more preferentially from 50% to 80% of the initial content.

### e) Concentration

The method according to the invention may further comprise a step e) of concentrating the clarified fraction using conventional water removal methods to obtain a concentrated aqueous extract.

Conventional water removal methods are known to the skilled person and include filtration, evaporation at atmospheric pressure or under vacuum, atomization, infrared drying, refraction window drying, and freeze-drying.

Preferentially, step e) of the method according to the invention enables the concentration of the molecules of interest while preserving the essential constituents of the clarified fraction.

Depending on the concentration method chosen, step of concentration e) removes all or some of the impurities present in the clarified fraction, such as solid residues, residual glycogen, oligomers and sugars resulting from glycogen degradation, in particular concentration by filtration.

Even more preferentially, the biomass treatment method according to the invention includes a step e) of concentrating the clarified fraction by filtration, in particular by tangential filtration such as ultrafiltration.

Preferentially, the biomass treatment method according to the invention comprises a step of concentration e) during which the clarified fraction is concentrated between 2 and 1000 times, even more preferentially between 20 and 60 times.

Preferentially, step e) is carried out at a pH of less than 7, preferentially between 1 and 6, even more preferentially between 2 and 5 and still more preferentially between 3 and 4.

In addition to further degradation by adding enzymes and/or the concentration of the clarified fraction, the method according to the invention can include subsequent steps for purifying the clarified fraction and/or the concentrated aqueous extract, in particular to purify the proteins in solution.

### Product According to the Invention

The product according to the invention comprises various organic materials depending on the treatment method applied, including water-soluble proteins including phycocyanins, sugars including glycogen degradation by-products (glucose oligomers) and possibly residual undigested glycogen and insolubles.

When present, phycocyanins may comprise phycocyanins resistant to acidic pH. Phycocyanins resistant to acidic pH are phycocyanins that are stable at acidic pH, that is they do not precipitate or lose their coloring at acidic pH. Resistance or stability to acidic pH can be measured as a loss of color of less than 10%, after a minimum of 10 minutes exposure to acidic pH, that is pH below 7, in particular pH between 2 and 5. Stability at acidic pH can also be measured by other methods, such as protein structure monitoring.

The presence of phycocyanins resistant to acidic pH results from carrying out steps d) and optionally e) at a pH below 7, in accordance with the extraction process described in WO2018/178334, preferentially at a pH between 1 and 6, even more preferentially between 2 and 5 and even more preferentially between 3 and 4.

According to a preferred mode, the product according to the invention comprises phycocyanins resistant to acidic pH.

A concentrated aqueous extract is obtained by the method according to the invention comprising a step e) of concentrating clarified fraction as described above. Conversely, a clarified fraction is obtained by the method according to the invention not comprising a step e) of concentrating clarified fraction as described above.

Preferentially, the product according to the invention comprises no exogenous enzymes or comprises a concentration of exogenous enzymes undetectable by conventional assay methods, notably after precipitation of proteins with acetonitrile, digestion with trypsin and then analysis by mass spectrometry (LC-MS-MS).

In particular, the product according to the invention preferentially comprises no exogenous enzymes selected from pectinases, amylases and pullulanases, or comprises a concentration thereof undetectable by conventional assay methods, in particular mass spectrometry.

If necessary, the product according to the invention is prepared in such a way as to eliminate any impurities that would make it unfit for consumption, in particular human consumption.

The product according to the invention can also be formulated by means known to the skilled person to avoid the degradation of its constituents during storage or subsequent use.

Finally, the product according to the invention, possibly formulated, can be packaged for storage and use, either in large-volume containers, or in smaller containers, for example with volumes corresponding to a single use, known as a single-dose, for human consumption. In this case, the container can be rigid, like a glass ampoule, or flexible, like a capsule suitable for consumption.

After acid hydrolysis of the sample, the sugar content, in particular glucose, mannose and galactose (referred to as “hydrolyzed glucose”, “hydrolyzed galactose” and “hydrolyzed mannose” respectively), of a product according to the invention is measured by high-performance liquid chromatography using a Hi-Plex H+ ion exclusion/ligand exchange column and refractometric detection (hereinafter referred to as “HPLC-RID determination of hydrolyzed sugars”). To do this, 1.5 mL of the supernatant from a vortex-homogenized sample is hydrolyzed with 1.5 mL of 2N sulfuric acid at 110° C. for 2 hours. The sample is then filtered (0.22 μm) and analyzed.

Note that the hydrolyzed glucose content measured in this way includes both the undigested residual glycogen glucose and the free glucose present in the product.

According to the invention, the free glucose content is determined by biochemical analysis, in particular using a YSI® biochemical analyzer following the manufacturer's recommendations.

Thus, to estimate the undigested residual glycogen content in a product according to the invention, simply subtract the free glucose content from the hydrolyzed glucose content.

The percentage of glycogen degradation is calculated as follows: (concentration of free glucose in the sample under study/maximum concentration of free glucose in an equivalent sample treated with 1% exogenous enzymes of the volume of enzyme solution relative to the total volume of the sample)×100.

According to the invention, the protein content of a product according to the invention is determined by the DUMAS method. The sample is subjected to high-temperature combustion in a stream of pure oxygen, and the nitrogen oxides produced are reduced by copper. After separation of the reaction by-products, nitrogen is measured using a thermal conductivity detector, and the result is expressed as N (%). This percentage of nitrogen is then reduced to a quantity of protein by applying the formula: N*6.25(%) (ISO/TS 16634-2:2009).

Finally, to determine the C-phycocyanin content of a sample according to the invention, 500 pl of sample are mixed with 100 mM Tris-Cl buffer pH 7.5 (1.5 ml) and absorbances at 652 and 620 nm measured using a Metier Toledo spectrophotometer. The C-phycocyanin concentration is then calculated using the following formula:

$$[ Phycocyanin ] in mg / mL = ( 0.162 \times A 620 nm - 0.098 \times A 652 nm ) \times Dilution$$

According to the invention, glycogen degradation of less than 10% by weight of total glycogen is not considered sufficient degradation. Moreover, degradation of less than 10% does not improve extract filtration. It is then considered that glycogen degradation is not functioning.

According to one embodiment, the clarified fraction obtained according to the invention has an estimated undigested residual glycogen (g/L)/C-phycocyanin (g/L) ratio of less than 6, advantageously less than 4, preferably less than 3, more preferentially less than 2.5, or even more preferentially less than 1.

The clarified fraction according to the invention has a total sugar concentration, measured by HPLC-RID determination of hydrolyzed sugars, of less than or equal to 40 g/L, in particular between 0.1 and 40 g/L, preferentially between 1 and 20 g/L, more preferentially between 3 and 11 g/L, and/or a hydrolyzed glucose concentration of less than or equal to 20 g/L, preferentially between 0.1 and 20 g/L, preferentially between 1 and 10 g/L, even more preferentially between 2 and 5 g/L and/or a hydrolyzed galactose concentration less than or equal to 10 g/L, in particular between 0.01 and 10 g/L, preferentially between 0.1 and 5 g/L, more preferentially between 0.5 and 3 g/L, and/or a hydrolyzed mannose concentration of less than or equal to 10 g/L, in particular between 0.01 and 10 g/L, preferentially between 0.1 and 5 g/L, more preferentially between 0.5 and 3 g/L, and/or a free glucose concentration of less than or equal to 10 g/L, in particular between 0.01 and 10 g/L, preferentially between 0.1 and 5 g/L, even more preferentially between 0.2 and 2 g/L.

The C-phycocyanin content of the clarified fraction advantageously ranges from 0.1 to 12 g/L, preferentially 0.5 to 8 g/L, more preferentially 1 to 7 g/L.

Preferably, the clarified fraction has a ratio (C-phycocyanins (g/L)/total sugars measured by HPLC-RID determination of hydrolyzed sugars (g/L)) of at least 0.001, preferentially 0.005 to 120, more preferentially 0.05 to 12, even more preferentially 0.1 to 3.

According to another preferred embodiment of the invention, the clarified fraction comprises C-phycocyanin and total sugars in a ratio (C-phycocyanin (g/L)/total sugars measured by HPLC-RID determination of hydrolyzed sugars (g/L)) of 0.005 to 120, in particular 0.05 to 12, more preferentially 0.1 to 3.

In a particular embodiment where the concentrated aqueous extract has a high C-phycocyanin concentration, the ratio (C— phycocyanin (g/L)/total sugars (g/L)) is at least 90, and may exceed 120.

Preferably, the clarified fraction has a ratio (C-phycocyanins (g/L)/hydrolyzed mannose (g/L)) of at least 0.01, preferentially 0.01 to 1200, more preferentially 0.1 to 80, even more preferentially 0.1 to 12.

Preferably, the clarified fraction has a ratio (C-phycocyanins (g/L)/hydrolyzed galactose (g/L)) of at least 0.01, preferentially 0.01 to 1200, more preferentially 0.1 to 80, even more preferentially 0.1 to 12.

The ratio (C-phycocyanin (g/L)/hydrolyzed glucose (g/L)) of the clarified fraction is preferentially less than 120, and preferentially between 0.005 and 120. Preferentially, this ratio is between 0.05 and 12, more preferentially 0.1 and 10.

Preferentially, the concentrated aqueous extract according to the invention has a dry matter content 2 to 1000 times higher than that of the clarified fraction from which it is derived, and even more preferentially 20 to 60 times higher.

The C-phycocyanin content of the concentrated aqueous extract advantageously ranges from 7 to 120 g/L, preferentially 20 to 100 g/L, more preferentially 40 to 80 g/L.

The concentrated aqueous extract according to the invention has a total sugar concentration, measured by HPLC-RID determination of hydrolyzed sugars, of less than or equal to 50 g/L, in particular between 1 and 50 g/L, preferentially between 5 and 40 g/L, more preferentially between 10 and 30 g/L, and/or a hydrolyzed glucose concentration of less than or equal to 20 g/L, preferentially between 0.1 and 20 g/L, preferentially between 1 and 10 g/L, even more preferentially between 2 and 5 g/L and/or a hydrolyzed galactose concentration less than or equal to 15 g/L, in particular between 0.5 and 15 g/L, preferentially between 1 and 13 g/L, more preferentially between 3 and 8 g/L, and/or a hydrolyzed mannose concentration of less than or equal to 15 g/L, in particular between 0.5 and 15 g/L, preferentially between 1 and 13 g/L, more preferentially between 3 and 8 g/L, and/or a free glucose concentration of less than or equal to 15 g/L, in particular between 0.01 and 15 g/L, preferentially between 0.5 and 8 g/L, even more preferentially between 1 and 4 g/L.

The ratio by weight (C-phycocyanin (g/L)/hydrolyzed glucose (g/L)) of the concentrated aqueous extract ranges from 2 to 80. Preferentially, this ratio is between 10 and 70, more preferentially 15 and 60.

Preferably, the concentrated aqueous extract has a ratio (C-phycocyanin (g/L)/hydrolyzed mannose (g/L)) of at least 0.01, preferentially 0.01 to 1200, more preferentially 0.1 to 80, even more preferentially 0.3 to 12.

Preferably, the concentrated aqueous extract has a ratio (C-phycocyanin (g/L)/hydrolyzed galactose (g/L)) of at least 0.01, preferentially 0.01 to 1200, more preferentially 0.1 to 80, even more preferentially 0.3 to 12.

Preferably, the concentrated aqueous extract has a ratio (proteins (%)/estimated undigested residual glycogen (g/L)) of 0.2 to 6.0, more preferentially 0.5 to 5.0, even more preferentially 1.0 to 4.5.

Preferably, the concentrated aqueous extract has an estimated undigested residual glycogen content (hydrolyzed glucose content (g/L)−free glucose content (g/L)) of 0.1 to 10 g/L, more preferentially 0.2 to 7 g/L, even more preferentially 0.3 to 5 g/L.

Preferably, the concentrated aqueous extract has a ratio (proteins (%)/total sugars measured by HPLC-RID determination of hydrolyzed sugars (g/L)) of less than 1%, preferentially between 1 and 0.01%, more preferentially between 0.8 and 0.05%, even more preferentially between 0.6 and 0.1%.

Finally, the present invention relates to a process for the preparation of a concentrated aqueous extract of a phycocyanin-producing microalgal ARU biomass having a high glycogen content, said process comprising a treatment method with a step of glycogen degradation, and a step of concentration as previously defined.

### Use

The invention also relates to the use of all or part of a product obtained from a method according to the invention as a product for the preparation of pharmaceutical or dietary compositions, including foods or pharmaceutical, nutraceutical, dietary, cosmetic or industrial compositions. In particular, the use of a product obtained according to a method of the invention comprising phycocyanins for the preparation of pharmaceutical or dietary compositions.

According to one embodiment, a composition according to the invention comprises all or part of a product obtained with the method according to the invention and one or more excipients.

According to a preferred embodiment, the composition according to the invention is an acidic composition whose pH is less than or equal to 6, preferably less than or equal to 5, even less than or equal to 4 and in particular between 2 and 4.

According to the invention, acidic composition means any composition comprising all or part of a product obtained with a method according to the invention as well as a mineral or organic acid.

Mineral or organic acids which may be used in the compositions according to the invention are known to the skilled person. Mineral acids include carbonic, phosphoric, hydrochloric, sulfuric, perchloric, sulfonic and nitric acids. Organic acids include citric, lactic, malic, tartaric and succinic acids.

Acidic compositions according to the invention can also comprise a vehicle which may comprise structural constituents associated with active compounds identified with regard to their nutritional contributions or for their properties beneficial to human or animal health.

Compositions other than dietary compositions may be pharmaceutical, veterinary or cosmetic, and may further comprise one or more additives and/or active ingredients known and used for this type of indication.

Compositions according to the invention can be presented in any usual form known to the skilled person. These include solid, liquid, fluid, pasty or viscous forms, in particular creams, gels, foams and pastes. Compositions according to the invention can also take the form of dry foods for cooking, powders for diluting, gelatinous compositions for food compositions.

In these solid compositions, all or part of the extract obtained according to the invention is preferably added in powder form. In this case, the product obtained from a method according to the invention is first dried to take the form of a powder.

The liquid compositions are advantageously aqueous compositions in which all or part of the extract obtained according to the invention is dissolved.

According to a particular embodiment of the invention, the liquid composition can be a dietary composition and more specifically an acidic, carbonated or non-carbonated beverage. Examples include sodas, juices, sports drinks, exercise drinks, energy drinks, etc. The compositions of these beverages are well known to skilled persons, and may comprise sugars, mineral salts, food additives or dissolved gas. An acidic beverage according to the invention can be a prior art acidic beverage in which the dye usually employed has been replaced in whole or in part by a product comprising acidic pH-resistant phycocyanins according to the invention.

The phycocyanin content in the compositions according to the invention will be in line with the concentrations usually applied in the intended field of application.

### EXAMPLES

### Example 1: Analysis of Glycogen Degradation with and without the Addition of Exogenous Enzymes at Different pH Levels

The amount of free glucose over time in Galdieria sulphuraria biomass lysates at pH 3.75, without enzyme addition, at pH 6 without enzyme addition and at pH 6 with enzyme addition (1% volume of enzyme solution to total lysate volume, “Amylase AG XXL” from Novozymes), at 37° C., was measured as a function of time (YSI® 2950).

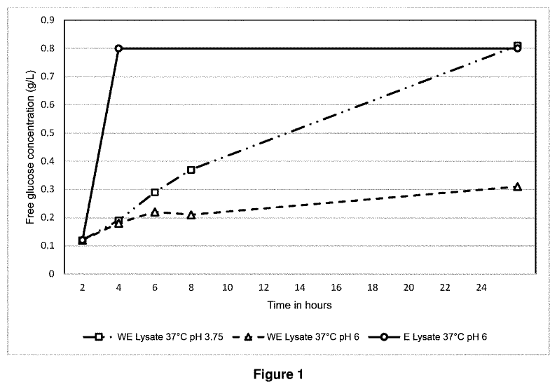

These kinetics are shown in FIG. 1.

Acidification of the lysate is achieved by adding a sufficient quantity of citric acid to change the pH from 6 to 3.75.

The results show that at acidic pH, 3.75, more glucose is released than at pH 6. After 24 hours incubation at pH 3.75 without enzyme addition, free glucose in the sample even reached the same level as in the sample to which exogenous enzymes had been added.

Note that glucose release also occurs at pH 6 in the absence of exogenous enzymes; the kinetics are just slower (gentler slope).

### Example 2: Analysis of Glycogen Degradation without the Addition of Exogenous Enzymes at Different pH Levels

The amount of free glucose over time in Galdieria sulphuraria biomass lysates (20% dry matter (DM) by weight of total lysate weight) at pHs between 3 and 7 without enzyme addition and at room temperature (20° C.) was measured over time (YSI® 2950).

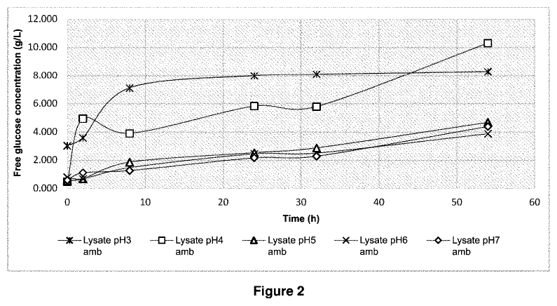

These kinetics are shown in FIG. 2.

Acidification and basification of the lysate are achieved by adding a sufficient quantity of citric acid or sodium hydroxide, respectively.

The results show that, over a given period of time, the higher the pH, the less glycogen is degraded. Glycogen digestion without the addition of exogenous enzymes on lysate shows activity mainly under the most acidic conditions. It can be seen here that this activity is high at pH 3 but slightly lower at pH 4. Activity at pH levels 5 to 7 is identical, but slower than at lower pH levels.

### Example 3: Analysis of Glycogen Degradation at Different Temperatures with and without the Addition of Exogenous Enzymes

The amount of free glucose over time in Galdieria sulphuraria biomass lysates (20% dry matter (DM) by weight of total lysate weight) at pH 3.75 is analyzed for different temperatures (4° C., 20° C. and 37° C.) without and with an enzyme (1% “Amylase AG XXL” from Novozymes) at pH 3.75 and at a temperature of 37° C. (YSI® 2950).

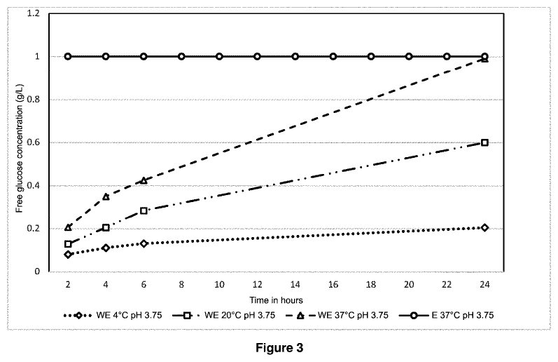

The results are presented in FIG. 3. They show a digestion of glycogen into free glucose even in the absence of enzyme addition. In particular, after 24 hours at 37° C., the amount of free glucose is the same with and without enzyme addition.

### Example 4: Analysis of Glycogen Degradation at Different Temperatures without the Addition of Exogenous Enzymes

The amount of free glucose over time in Galdieria sulphuraria biomass lysates (20% dry matter (DM) by weight of total lysate weight) at pH 4 is analyzed for different temperatures (4° C., 15° C., 20° C., 30° C., 40° C. and 50° C.) without the addition of exogenous enzymes (YSI® 2950).

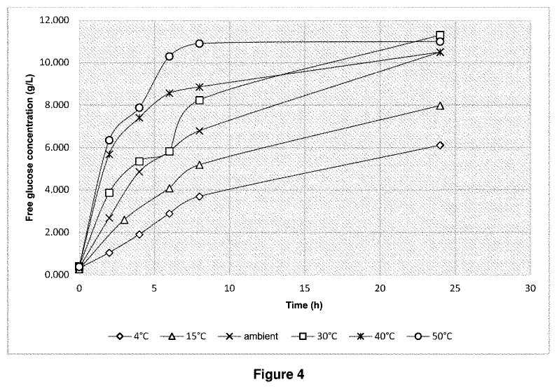

The results are presented in FIG. 4. They show that, over a given time, the higher the temperature, the greater the amount of glycogen digested during a step of rest according to the invention.

### Example 5: Analysis of Glycogen Degradation at Different Stages of an Extraction Method

The amount of free glucose, at room temperature (20° C.), in lysate (20% dry matter (DM) by weight relative to the total weight of the lysate) and Galdieria sulphuraia biomass solubilizate (7.5% DM by weight relative to the total weight of the solubilizate) at pH 3.75, without the addition of exogenous enzymes, is analyzed (YSI® 2950). In this context, the kinetics of glucose release at room temperature (20° C.), in Galdieria sulphuraria biomass lysate at pH 3.75 with the addition of exogenous enzymes (1% of the volume of enzyme solution in relation to the total volume of lysate “Amylase AG XXL”, Novozymes) is taken as a control.

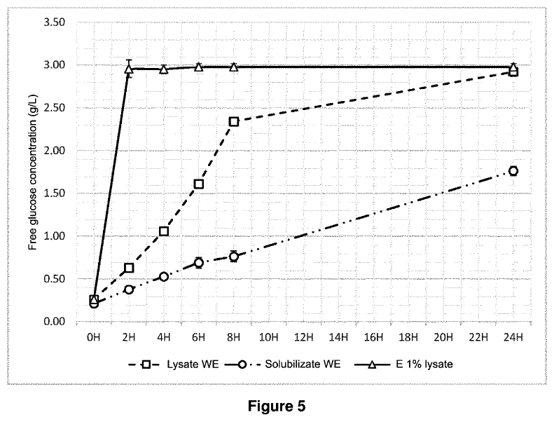

These kinetics are shown in FIG. 5.

From the results obtained and by extrapolation, it can be concluded that, even without enzyme addition, in the case of lysate, after 24 hours all digestible glycogen will be degraded and released as free glucose. In the case of solubilizate, on the other hand, the reaction seems slower and is still not complete after 24 hours.

It should be noted that the samples studied all showed a glycogen degradation of over 10%, and thus good filterability after glycogen digestion, whether they were prepared with or without the addition of exogenous enzymes.

### Example 6: Analysis of Glycogen Degradation in the Prior Art

In patent WO2020/144330, Example 2 shows the degradation of glycogen in a crude solution of phycocyanins in the presence and absence of exogenous enzymes.

To achieve this, Galdieria sulphuraria biomass is produced and then recovered/harvested. The cells are then lysed and diluted to obtain a solubilized aqueous extract. A clarified fraction is then obtained from this aqueous extract by separating the solids through filtration on a 0.22 μm filter.

The amount of free glucose, at room temperature (20° C.), in Galdieria sulphuraria biomass clarified fraction (7.5% MS by weight with respect to the total weight of the clarified fraction) at pH 4, without and with a step of adding exogenous enzymes (0.1% of the volume of enzyme solution in relation to the total volume of clarified fraction “Amylase AG XXL” from Novozymes, “Pectinex Ultra SP-L” from Novozymes or “BAN 480 L” from Novozymes) is analyzed (YSI 2700 Biochemistry Analyzer).

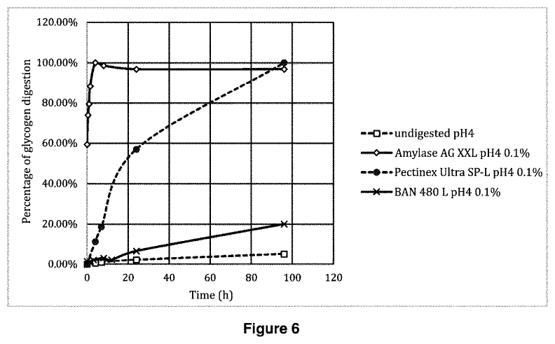

The results are presented in FIG. 6.

From the results obtained, it can be concluded that without the addition of exogenous enzymes, and with the clarified fraction kept at rest for 95 hours, glycogen is still not sufficiently digested (less than 10% degradation). However, with the addition of an enzyme, after 95 hours, all the glycogen is digested.

### Example 7: Analysis of Glycogen Degradation at all Stages of an Extraction Method

The amount of free glucose, at room temperature (20° C.), in lysate (20% DM by weight relative to the total weight of the lysate), solubilizate, and clarified fraction of Galdieria sulphuraria biomass (7.5% DM by weight relative to the total weight of the solubilizate or clarified fraction) at pH 4, without the addition of exogenous enzymes, is analyzed (YSI® 2950). The clarified fraction is obtained after filtration of the solubilizate through a 0.22 μm filter.

In this context, glucose release kinetics at room temperature (20° C.), in lysate, solubilizate and clarified fraction of Galdieria sulphuraria biomass at pH 4 with the addition of exogenous enzymes (1% of enzymatic solution volume with respect to total lysate volume “Amylase AG XXL”, Novozymes) are used to determine the maximum free glucose concentration of the sample.

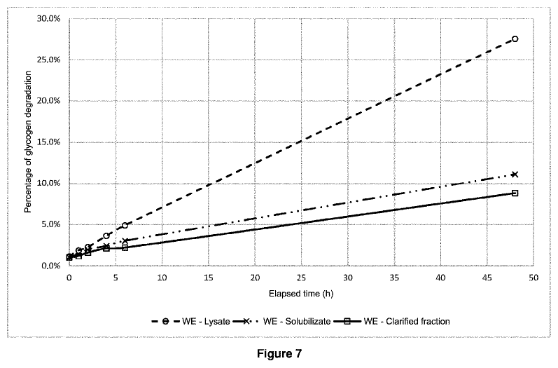

These data were used to calculate the percentages of glycogen degradation (digestion) shown in FIG. 7.

From the results obtained, we see that, without the addition of exogenous enzymes, after 48 hours, more than 10% of glycogen is digested when lysate and solubilizate are kept at rest, but not the clarified fraction.

### Example 8: Characterization of an Extract Prepared According to the Invention

A Galdieria sulphuraria biomass lysate (20% DM by weight of total lysate weight) is kept at an acidic pH of 3.7 for 12 hours at 25° C.

An extract is prepared from this lysate. To achieve this, the lysate is washed with a quantity of water representing less than 4 times the total volume of lysed biomass. This volume of water is divided into 3 fractions to perform 3 successive washes of the lysed biomass. Washing water is recovered, in accordance with the teaching of patent application WO2020/161280. The product obtained is then filtered on a hollow fiber membrane with a porosity of 70 kDa (clarified fraction) with a final diafiltration step and the filtrate is recovered, in accordance with the teaching of patent application WO2020/144330 (concentrated aqueous extract).

The aqueous extract concentrate and clarified fraction are analyzed for total sugars, hydrolyzed mannose, hydrolyzed galactose and hydrolyzed glucose by HPLC-RID, free glucose by YSI®2950, phycocyanin and protein.

The results obtained are presented in Table 1 below.

TABLE 1

| | Clarified | Concentrated |
|---|---|---|
| | fraction | aqueous extract |
| Free glucose (g/L) | ND | 0.04 |
| Hydrolyzed glucose (g/L) | 3.40 | 1.5 |
| Hydrolyzed galactose (g/L) | 0.90 | 7.00 |
| Hydrolyzed mannose (g/L) | 0.95 | 8.00 |
| C-phycocyanin (g/L) | 6.88 | 78.00 |
| Ratio of proteins (%) to estimated | ND | 4.3 |
| undigested residual glycogen (g/L) | | |
| Ratio of proteins (%) to total sugars (g/L) | ND | 0.4 |

1. A method for treating a biomass of unicellular red algae (URA) of the genus Galdieria, said URA biomass treatment method comprising the steps of:
a) harvesting of biomass by separating the culture medium to obtain a raw biomass of URA,
b) cell lysis of the crude biomass from step (a) to obtain a lysate;
c) optional dilution of the lysate from step (b) to obtain a solubilizate; and,
d) separation of insolubles suspended in the lysate from step (b) or the solubilizate from step (c) to obtain a clarified fraction, further comprising a step of glycogen degradation in which the raw biomass and/or lysate and/or if applicable solubilizate is kept at rest (step of rest) for at least 3 hours, during which the liquid medium comprising glycogen is at acidic pH.

2. The method according to claim 1, wherein the step of rest lasts between 3 hours and one week.

3. The method according to claim 1, wherein the step of rest takes place at a temperature of between 15° C. and 70° C.

4. The method according to claim 1, wherein the step of rest is carried out on the lysate and/or solubilizate if applicable, preferentially on the lysate.

5. The method according to claim 1, wherein it comprises, prior to the step of rest, a step of adjusting the pH of the raw biomass, lysate and/or if applicable solubilizate to a pH below 7, in particular between 1 and 6, preferentially between 2 and 5, more preferentially between 3 and 4.

6. The method according to claim 1, wherein the method further comprises a step e) of concentrating the clarified fraction to obtain a concentrated aqueous extract.

7. A product obtainable by the method according to claim 1.

8. The product according to claim 7 comprising an estimated undigested residual glycogen content of 0.1 to 10 g/L, more preferentially 0.2 to 7 g/L, even more preferentially 0.3 to 5 g/L.

9. The product according to claim 7, comprising a hydrolyzed mannose concentration less than or equal to 15 g/L.

10. The product according to claim 7 comprising a hydrolyzed galactose concentration less than or equal to 15 g/L.

11. The product according to claim 7 comprising a protein (%)/undigested residual glycogen ratio estimated by HPLC-RID determination of hydrolyzed sugars (g/L) of 0.2 to 6.0, more preferentially 0.5 to 5.0, even more preferentially 1.0 to 4.5.

12. The product according to claim 7 comprising, a protein (%)/total sugars ratio measured by HPLC-RID determination of hydrolyzed sugars (g/L) of less than 1%, preferentially between 1 and 0.01%, more preferentially between 0.8 and 0.05%, even more preferentially between 0.6 and 0.1%.

13. The product according to claim 7 comprising a C-phycocyanin content of between 0.1 and 20 g/L.

14. A nutraceutical, dietary, or cosmetic composition comprising a product according to claim 7.

15. The composition according to claim 14 comprising a mineral or organic acid.

16. A nutraceutical, dietary, or cosmetic composition comprising a product according to claim 13.

17. The composition according to claim 16 comprising a mineral or organic acid.
